(19) United States
(12) Patent Application Publication
(10) Pub. No.: US 2024/0395370 A1
(43) Pub. Date: Nov. 28, 2024

(54) METHODS FOR DETECTING THE PRESENCE OF BACTERIA, FUNGI, PARASITES, AND VIRUSES IN A TEST SAMPLE AND MONITORING AND TREATING A PATIENT

(71) Applicant: Deborah Ann Payne, Lakewood, CO (US)

(72) Inventor: Deborah Ann Payne, Lakewood, CO (US)

(21) Appl. No.: 18/584,557

(22) Filed: Feb. 22, 2024

Related U.S. Application Data
(63) Continuation-in-part of application No. 16/831,546, filed on Mar. 26, 2020, now abandoned.

(51) Int. Cl. G16H 10/40; C12Q 1/689; C12Q 1/6895

(52) U.S. Cl. CPC G16H 10/40; C12Q 1/689; C12Q 1/6895; C12Q 2600/158

(57) ABSTRACT

The present invention relates to detection of bacteria, fungi, parasites, and viruses and, more specifically to methods for detecting the presence of microorganisms/viruses associated with certain diseases or infections, including but not limited to sexually transmitted infections, bacterial vaginosis, aerobic bacterial vaginitis, and vulvovaginal candidiasis.

### CROSS REFERENCE TO RELATED APPLICATIONS

This application claims priority to and is a continuation-in-part of co-pending U.S. patent application Ser. No. 16/831,546, entitled METHODS FOR DETECTING THE PRESENCE OF BACTERIA, FUNGI, PARASITES, AND VIRUSES IN A TEST SAMPLE AND TREATING A PATIENT, filed on Mar. 26, 2020; and provisional patent application Ser. No. 62/824,348, entitled METHODS FOR DIAGNOSING THE PRESENCE OF BACTERIA IN A TEST SAMPLE, filed Mar. 27, 2019; the entire contents of which are hereby incorporated by reference in their entirety for all purposes.

### SEQUENCE LISTING XML

This application is being filed on or after Jul. 1, 2022, and contains a sequence disclosure. The XML Listing File name is “DP-101-CIP.xml”. The XML file was created on Aug. 13, 2024, with the original file data created on Feb. 25, 2020. The XML file size is 84 kb. The entire contents of the Sequence Listing XML are hereby incorporated by reference in their entirety for all purposes.

### FIELD OF THE INVENTION

The present invention relates to detection of bacteria, fungi, parasites, and viruses and, more specifically to methods for detecting the presence of microorganisms/viruses associated with certain diseases or infections, including but not limited to sexually transmitted infections, bacterial vaginosis, aerobic bacterial vaginitis, and vulvovaginal candidiasis.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND OF THE INVENTION

Bacterial vaginosis (BV), aerobic vaginitis (AV) and vulvovaginal candidiasis (VVC) are associated with a perturbation of the normal vaginal microbiota. The bacteria and yeast associated with these conditions may be commensal or contaminants from the gastro-intestinal tract that increase in number relative to lactobacilli species that are associated with a healthy microbiota. Healthy bacteria such as lactobacilli can protect the vaginal tract from sexually transmitted infections (e.g. Trichomonas vaginalis).

Bacterial Vaginosis (BV) is the most common cause of abnormal vaginal discharge in women of childbearing age. It may also be encountered in menopausal women and is very rare in children. The prevalence of BV by race is: Caucasian women: 5-15%, Black women: 45-55%, and Asian women: 20-30%. Approximately 50% of BV is asymptomatic and is characterized by the overgrowth of predominately anaerobic bacteria (e.g. Gardnerella vaginalis, Mycoplasma hominis, Mobiluncus sp, etc.) in the vagina leading to a replacement of Lactobacilli sp and an increase in vaginal pH.

Molecular methods, such as polymerase chain reaction (PCR), have demonstrated a correlation between the presence of Bacterial Vaginosis Associated Bacteria (BVAB2) and Megasphaera-1 DNA and BV. Although not considered a sexually transmitted infection, BV is associated with sexual activity. The clinical criteria (also known as the Amsel criteria) for the diagnosis of BV includes: presence of foul fishy smell—whiff test on speculum and amine odor test on slide, thin white homogenous discharge coating walls and vestibule of vagina, vaginal pH greater than 4.5 and/or the presence of clue cells. While nitroimidazoles are frequently used to treat BV, A. vaginae and Mobiluncus species have been shown to possess resistance to such compounds and may respond better to lincosamides.

Aerobic vaginitis (AV) is a vaginal condition characterized by inflammation of the vagina and colonization by aerobic microflora, such as Escherichia coli, Group B Streptococcus and other Streptococus spp., Staphylococcus aureus, Staphyloccus epidermidis, and Enterococcus faecalis. Symptoms may also include purulent yellow discharge, foul odor (though not ‘fishy’, as with bacterial vaginosis), small or large vaginal ulcerations, and complaints of vaginal dyspareunia. Aerobic vaginitis is a distinct clinical presentation from bacterial vaginosis, which is characterized by the absence of inflammation and colonization by predominantly anaerobic organisms. In addition to having different clinical signs, aerobic vaginitis requires different treatment than bacterial vaginosis as these bacteria are aerobic or facultative anaerobes.

Vulvovaginal candidiasis (VVC) is caused by the overgrowth of fungal organisms, with most infections caused by Candida albicans (i.e., 90%). Candida glabrata is the second most frequent cause of Candida vaginitis. Other species, including Candida parapsilosis, Candida tropicalis, Candida dubliniensis, Candida krusei, and Candida kefyr, can also cause infection, particularly in immunocompromised and HIV-infected patients. C. parapsilosis is occasionally isolated from patients with VVC and is a significant cause of horizontally transmitted infections in neonatal hospital wards. C. kefyr is rarely identified in VVC but is considered an emerging pathogen in patients with hematologic malignancies. Candidiasis caused by C. glabrata and secondary infections caused by C. tropicalis are difficult to treat in part because they are often resistant to azole compounds such as fluconazole.

Clinical diagnosis and treatment of BV, AV, and VVC are relatively successful in the initial cure of the infection. However, clinical relapse rates after the initial treatment is significant between the various infections. The normalized results can be graphically displayed over a course of time to monitor for signs of clinical relapse or for success of the initially prescribed cure or wellness. A course of time varies depending on the case, initial treatment, prescribed cure, or wellness, to name a few. Studies have shown that over 50% of BV cases will recur within 12 months of the initial treatment. For VVC, over 20% of women who report an initial vaginal yeast infection will also report 4 or more infections within the following 12-month period. Four or more recurring yeast infections within a 12-month period is clinically defined as Recurrent Vulvovaginal Candidiasis (RVVC).

One of the main culprits of clinical relapse is the lack of molecular testing for the aforementioned infections which normalize test results for specific cases. The invention as described here has the purpose of mitigating recurring infections in cases of BV, AV, and VVC by normalization of results. Algorithmic normalization of data based on patient demographics provides for novel and more accurate diagnosis of vaginal colonizations, including vaginal infections. Patient demographics such as demographic information, infectious disease, prior infections, non-human pathogens such as bacteriophages, gender, age, biogeographical ancestry, pregnancy status, treatment, location, zip code, sexual activity, transgender status, number of different sexual partners over a predetermined period of time, sexual preference, sexual practices, smoking status, hormone replacement recipient, menopause, dental dams, contraceptive use, or type of condoms may affect interpretation of laboratory results. The virtual database or vaginal wellness can assist in clinical decision making. The database described here can be applied as an adjunctive test to current IVD tests. The normalization of results provides personalized diagnosis to patients with their test results regarding their personal sensitivity and specificity. A biomarker is used as an adjunctive test for IVDs for sexually transmitted infections, the virtual vaginal health database/interpretative tool, and a wellness test.

Normalization of data is required to produce accurate results. As previously mentioned, the prevalence of BV is higher in women of certain races than women of other races¹². Failure to normalize results according to biogeographical ancestry will provide less accurate diagnosis than a diagnostic algorithm which does normalize results. This is true for a myriad of other factors. Patients who are pregnant display differing bacteria than patients who are not pregnant. A patients age plays a role in differing estrogen levels, which in turn affects mucosal pH that permits anaerobic bacteria and yeast to grow more easily. The presence of non-human pathogens such as bacteriophages may be detrimental to healthy levels of Lactobacillus, which in turn may lead to BV. ¹https://www.cdc.gov/std/bv/stats.htm #:˜:text=Prevalence%20of%20BV%20increases%20based,than%20white%20women%20(23%25)²https://journals.lww.com/stdjournal/Fulltext/2007/11000/The_Prevalence_of_Bacterial_Vaginosis_in_the.6.aspx

Current IVD tests (e.g. Roche Cobas, Hologies Aptima, BD Affirm VPIII) for CT/NG/Trich do not contain a sample collection control that assures that adequate collection has occurred. IVDs for sexually transmitted diseases (Chlamydia, Gonorrhea, Trichomonas, Mycoplasma genitalium) include no tests for BV or aerobic vaginitis that include a cellularity control to confirm sample collection adequacy or aid in the interpretation of the results. It is not uncommon for the laboratory to receive a vial that has no specimen in it. The medical lab assistants pre-label samples for efficiency, but if the patient does not arrive at her appointment, the lab often receives and processes these samples. Moreover, a three-order of magnitude difference exists in how samples are collected by healthcare providers.

Other applications of this invention include providing improved quality assurance normalizing the detection of organisms against the host marker. Further applications permit the quantitative measurements of organisms that permit therapeutic monitoring of response to treatment. Additionally, self-collected samples from patients will have the benefit of this additional normalization to better ensure that inadequate samples are identified.

While C. krusei is not a frequent cause of candidiasis, it can also be resistant to azole compounds, including fluconazole, and shows intermediate resistance to amphotericin B. C. dubliniensis and C. kefyr are initially susceptible to commercially available antifungals, but have been observed to rapidly develop resistance to fluconazole and other classes of antifungal therapies following exposure to the drug. Increases in VVC caused by resistant Candida species have been observed with increased access to over the counter (OTC) azole medications. Therefore, correctly identifying the causative agents in cases of VVC and other infections is important for determining the course of treatment. Both C. glabrata and C. tropicalis can be differentiated from C. albicans and other Candida species using a variety of biochemical assays. Because these identification methods require viable organisms, molecular techniques provide additional capabilities to identify nonviable Candida organisms (i.e., from fixed samples).

While Candida dubliniensis is difficult to distinguish from C. albicans due to phenotypic and genotypic similarities, molecular testing can also provide an accurate and rapid distinction of Candida species. Criteria for the clinical diagnosis of candidiasis include: absence of smell (whiff test on speculum and in amine odor test on slide), which is supportive of candidiasis, but is not diagnostic. Patients with RVVC are recommended to have at least one speciation test. Risk factors (e.g., diabetes, underlying immunodeficiency, corticosteroid use, or frequent antibiotic use) should be excluded.

Complications of BV and AV in pregnant women are preterm delivery, neonatal sepsis, chorioamnionitis, and preterm membrane rupture. Yeast infections in pregnant women which may cause systemic infections in neonate particularly with low birth weight (LBW) and prematurity after delivery. Candidiasis is an important risk factor of systemic infections in low birth weights infants and the related mortalities.

BV may also be associated with surgical site infections and the American College of Obstetrics and Gynecology (ACOG) recommends that BV is treated prior to surgical procedures. Complications of BV and AV in non-pregnant patients include PID and increased risk for sexually transmitted infections such as HIV. Relapse for treatment of BV is frequent and occurs in more than 35% of cases.

Most infectious markers are normalized based on volume of blood, urine, serum and plasma for the collection. The reference diagnostic method for BV is gram stain method followed by scoring for the presence and absence of various bacteria (specifically, Lactobacillus sp) known as the Nugent score. Microscopic diagnosis of AV identifies a decrease in Lactobacillus spp., the presence of parabasal epitheliocytes (indicative of intense inflammation), and an increase in leukocytes and leukocyte toxicity using the Donders score. While concomitant anaerobic-aerobic conditions may exist, AV is usually associated with intermediate BV Nugent Scores. Certain oncology markers are normalized against a host marker such as BCR-ABL for chronic myelogenous leukemia. Some tests are available for confirming the human component for cervical cancer that targets Human Papillomavirus, but it is more a quality control measure than used for interpreting cutoffs. HPV is a tumor marker for cervical cancer screen with the final diagnosis being reflexed to histology from biopsies collected from the patient.

For candidiasis, examination for yeasts or pseudohyphae on wet mounts and on gram stains have sensitivities of 40-60% and 65%, respectively. Culture directly onto Sabouraud's plate, reported as light, medium, and heavy growth, which correlates with specificity. However, culture is not possible following fixation into liquid based cytology medias. Molecular methods that quantify the presence and absence of these bacteria are available for BV, AV and candidiasis. These methods can be deficient as ascertaining a quantitative value of the presence of the bacteria or Candida sp of interest can be skewed if a larger or smaller sample than normal is taken.

Some sexually transmitted infections caused by such as Trichomonas sp, Chlamydia trachomatis, Neissera gonorrhoeae, Mycoplasma genitalium and by viruses (i.e., Herpes simplex virus 1 and 2, Varicella Zoster Virus) are found in vaginal and other sample sources. It should be understood that such infections can also be found in other sites, including but not limited to rectal and pharyngeal. Moreover, such infections are not necessarily confined to women; man also may have infections. All of the foregoing are therefore within the scope of the present invention.

### DESCRIPTION OF RELATED ART

U.S. Pat. No. 9,057,111 issued to Cartwright, et al. for DIAGNOSTIC METHODS AND MARKERS FOR BACTERIAL VAGINOSIS on Jun. 16, 2015, discloses a method of diagnosing bacterial vaginosis in a woman, which involves determining an amount of each of more than one BV-associated bacterium in a vaginal sample obtained from the female and assessing a BV status of the female based on the amount of each of the more than one BV-associated bacterium in the sample.

U.S. Pat. No. 9,200,331 issued to Johnson, et al. for METHODS FOR THE DIAGNOSIS OF BACTERIAL VAGINOSIS on Dec. 1, 2015, discloses methods for the diagnosis of bacterial vaginosis based on an analysis of a patient sample. For example, patient test samples are analyzed for the presence or absence of one or more lactobacilli and two or more pathogenic organisms. The presence or absence of one or more lactobacilli and two or more pathogenic organisms may be detected using PCR analysis of nucleic acid segments corresponding to each target organism. The quantity of the target organisms can then be used to determine a score which is indicative of a diagnosis of bacterial vaginosis.

U.S. Pat. No. 9,624,552 issued to Cartwright, et al. for DIAGNOSTIC METHODS AND MARKERS FOR BACTERIAL VAGINOSIS on Apr. 18, 2017, discloses a method of diagnosing bacterial vaginosis in a woman, which involves determining an amount of each of more than one BV-associated bacterium in a vaginal sample obtained from the female and assessing a BV status of the female based on the amount of each of the more than one BV-associated bacterium in the sample.

U.S. Pat. No. 10,131,959 issued to Cartwright, et al. for METHODS FOR THE DIAGNOSIS OF BACTERIAL VAGINOSIS on Nov. 20, 2018, discloses a method of diagnosing bacterial vaginosis in a woman, which involves determining an amount of each of more than one BV-associated bacterium in a vaginal sample obtained from the female and assessing a BV status of the female based on the amount of each of the more than one BV-associated bacterium in the sample.

None of the foregoing patents normalizes the detection against a human biomarker or database for quantitative or qualitative results. Nor do any of the foregoing references address aerobic vaginitis, fungal, parasites, viral, STI or wellness scores.

### SUMMARY OF THE INVENTION

In accordance with the present invention, there is provided a method of testing for the presence of an infectious biomarker. A test sample comprising cellular and non-cellular material is collected from a patient, the test sample having a first amount of at least one infectious biomarker and a second amount of a host biomarker(s). The amount of the infectious biomarker(s) in the test sample is determined as is the amount of the host biomarker. The amount of infectious biomarker(s) in the test sample is normalized as a function of the amount of host biomarker therein. Normalization of the results is comprised by a database of algorithms which characterize specimens according to a variety of factors, including patient demographics. The normalization can account for relative diagnosis scores such as the Nugent, Donder, and the scoring of fungal elements per high power field or manual counting using hemocytometry linked to molecular detection results. Also included in the database is quantitative data derived from molecular methods. The normalizing step is a ratio of the first amount of the infectious biomarker(s) to the second amount of the host biomarker. The test sample is positive for a disease associated with the infectious biomarker(s) if the ratio is above a predetermined value, commonly referred to as the threshold cycle value. The normalized results will utilize information in the database to assign specificity and/or sensitivity values from the patient. These values are fitted to the RoC curve as a logistic regression model to classify observations of the results as either a true positive or false positive rate.

Infectious biomarkers in different embodiments can be associated with harmful microorganisms (i.e., fungi, parasites, viruses) as well as beneficial bacteria such as lactobacillus or other “good” bacteria (i.e., non-harmful bacteria).

In some embodiments, normalizing the first amount of the infectious biomarker can include determining a ratio of the first amount of the infectious biomarker to the second amount of the host biomarker. The method can further include the step of identifying the test sample as being positive for disease associated with the infectious biomarker associated if the ratio is above a predetermined value, and identifying the test sample as being negative for disease associated with the infectious biomarker if the ratio is below the predetermined value. Normalized data can be used to trend the quantity of pathologic organisms or “good” bacteria from a patient over time. Likewise, such normalized data can be used to trend parasitic organisms (e.g. Trichomonas sp.) or fungal infections such as candidiasis or viruses.

One objective of the present disclosure is to help normalize the results of specimen collection and data obtained therefrom, provide specificity data that will inform the healthcare provider on the relative quantity of organisms detected. Therapy may be tailored to organisms associated with high specificity. For example, nitroimidazoles preserve lactobacilli species that are beneficial organisms yet will not be affective against Atopobium vaginae, Mobiluncus sp, Mycoplasma hominis, Ureaplasma urealyticum or Ureaplasma parvum (and U. parvum genotypes). Clindamycin is more effective against Atopobium vaginae, Mobiluncus curtisii and Mycoplasma hominis but also will kill beneficial Lactobacilli sp. Therefore, the specificity score may assist in staging drug treatment.

Yet another objective of the invention is to use normalized data to assign a wellness score for the presence of Lactobacillus sp or other biomarker(s). The value of multiple results from various infectious entities can be used to generate an interpretative result. The example of the Lactobacillus can be used as a “wellness metric” as the presence of these bacteria is protective against BV, AV, and candidiasis.

### BRIEF DESCRIPTION OF THE DRAWINGS

A complete understanding of the present invention may be obtained by reference to the accompanying drawings, when considered in conjunction with the subsequent detailed description, in which:

FIG. 1 is a table of primer and probe sequences for human and some bacteria associated with vaginal bacterial dysbiosis;

FIG. 2 is a table of primer and probe sequences for the bacterial order Mycoplasmatoales vaginal dysbiosis;

FIG. 3 is a table of examples of primers and probes for the detection of microorganisms associated with aerobic vaginitis;

FIG. 4 is a table of examples of primers and probes associated with healthy or vaginal wellness for Lactobacillus sp;

FIG. 5 is a table of examples of primers and probes for the detection of fungal microorganisms;

FIG. 6 is a table of examples of primers and probes for the detection of viruses and protozoan associated with vaginal infections;

FIG. 7 is a graph illustrating a range of DNA collected from patients for testing;

FIG. 8 is a table illustrating examples of results with diagnostic values that are pulled from the virtual database of tested samples;

FIG. 9 is a Receiver Operator Curve (RoC) for an algorithm for Atopobium vaginae without normalization with a host biomarker;

FIG. 10 is a Receiver Operator Curve (RoC) using a host and infectious organism (e.g., Atopodium vaginae) biomarker algorithm;

FIGS. 11a and 11b depict graphs showing decreasing levels of infectious agent for serially collected samples with constant levels of a host biomarker with and without normalizing the infectious agent with the host biomarker;

FIG. 12a is a graph illustrating examples of constant levels of infectious agent without normalization against a host biomarker;

FIG. 12b is a graph illustrating the inclusion of a host biomarker data algorithm, indicating an increase in the relative amount of infectious agent;

FIG. 13a is a graph that illustrates decreasing amounts of an infectious agent without normalization with a host biomarker over time;

FIG. 13b is a graph depicting decreasing levels of an infectious agent over time with the data normalized with a host biomarker algorithm;

FIG. 14a is a graph illustrating variable infectious agent detection without normalization to variable host biomarker;

FIG. 14b is a graph illustrating algorithm of variable agent normalized by variable levels of host biomarker with diagnostic cutoff designation (flat line);

FIG. 15 is a portion of a table derived from a database showing sensitivity with specificity;

FIG. 16 is a flowchart demonstrating the development and implementation of a virtual vaginal wellness database;

### DETAILED DESCRIPTION OF THE PREFERRED EMBODIMENTS

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

The invention is a method of testing for the presence of an infectious biomarker. The amount of the infectious biomarker(s) in the test sample is determined as is the amount of the host biomarker. The test sample is positive for a disease associated with the infectious biomarker(s) if the ratio of the amount of the infectious biomarker(s) to the amount of the host biomarker is above a predetermined value.

The invention provides methods for diagnosing bacterial vaginosis (BV), aerobic vaginitis (AV), vulvovaginal candidiasis (VVC), parasite, STI, or viral conditions by quantitatively detecting amplified nucleic acids from microorganisms/viruses and normalizing the data by using quantitative methods for detection of amplified human nucleic acid and/or healthy bacteria. The normalized information is used to confirm specimen adequacy and to determine if a person has bacterial vaginosis (BV), aerobic vaginitis (AV), vulvovaginal candidiasis (VVC), and for STI testing. The normalized data can be used to diagnose individuals suffering from these conditions, to stage therapeutic treatment based on specificity and diagnostic accuracy data and monitor treatment response over time.

Techniques in molecular biology, cell biology, immunology, microbiology, bioinformatics, database analysis and statistical modeling are used in practicing the inventive method.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed.

The term “amplification” or “amplify” means increasing the number of copies of a target nucleic acid. A target may be either DNA or RNA. The examples described here utilize polymerase chain reaction but other methods for amplifying and detecting nucleic acids are known.

The terms “sensitivity” (also called the true positive rate) measures the proportion of actual positives that are correctly identified as such (e.g., the percentage of sick people who are correctly diagnosed using the test as having bacterial vaginosis, aerobic vaginitis or vulvovaginal candidiasis). The formula used for this determination is:

$$\frac{True Positive}{( True Positive + False Negatives )}$$

The term “specificity” (also called the true negative rate) measures the proportion of actual negatives that are correctly identified as such (e.g., the percentage of healthy people who are correctly identified as not having bacterial vaginosis, aerobic vaginitis or vulvovaginal candidiasis). The formula used for this determination is:

$$\frac{True Negative}{( True Negative + False Positive )}$$

“True positive” means sick people correctly identified as sick.
“False positive” means healthy people incorrectly identified as sick.
“True negative” means healthy people correctly identified as healthy.
“False negative” means sick people incorrectly identified as healthy.

“Receiver operator characterization or curve” (RoC) is a graphical plot illustrating the diagnostic ability of a binary classifier system as its discrimination threshold is varied. RoC and other statistical programs are readily available to determining sensitivity and specificity (e.g., see https://www.medcalc.org/contact.php). Alternatively, a graphical description of test performance representing the relationship between the true-positive fraction (sensitivity) and the false-positive fraction (1-specificity) can be provided.

“Statistical accuracy of measurement” indicates the closeness of agreement between a measured quantity value and a true quantity value of a measurement and is mathematically defined as:

$$\frac{( True Positive + True Negatives )}{( True Positive + True Negative + False Positive + False Negative )}$$

“Reference method” is an exactly-defined technique used in association with an internationally agreed reference preparation to provide sufficiently precise and accurate data for assessing the validity of other methods (modified from ICSH).

“Database” refers to a structured set of data retained on a computer. The database used in this process is known as Access™, provided by Microsoft Office 365, but other database systems are available. Briefly, residual specimens were retained for microscopic analysis following routine processing for molecular detection. Conventional scoring using microscopy is used to characterize specimens according the Nugent score, Donder, scoring of fungal elements per high power field or manual counting using hemocytometry linked to molecular detection results with patient demographic data. The database includes quantitative data derived from molecular methods.

The “Ct or threshold cycle value” is the cycle number at which the fluorescence generated within a reaction crosses the fluorescence threshold, a fluorescent signal significantly above the background fluorescence. At the threshold cycle, a detectable amount of amplicon product has been generated during the early exponential phase of the reaction. The threshold cycle is inversely proportional to the original relative expression level of the gene of interest.

“Host” is defined as the entity from which the specimen is collected.

“Biomarker” is defined as a specific analyte (DNA, RNA, protein) found in a patient specimen or sample that is useful for measuring the progress of disease or the effects of treatment.

“Specimen” is defined as discrete portion of a body fluid, breath, hair, or tissue taken for examination, study, or analysis of one or more quantities or properties assumed to apply for the whole (ISO 15189). Alternatively, “specimen” can refer to a discrete portion of a body fluid or tissue taken for examination, study, or analysis of one or more quantities or characteristics to determine the character of the whole.

A “primary sample collection device” is an apparatus specifically intended by an IVD manufacturer to obtain, contain, and preserve a body fluid or tissue for in vitro diagnostic examination.

“Analyte component” is represented in the name of a measurable quantity.

“Liquid with preservative qualities” is defined as a media that deters or prevents the degradation of biomolecules either by inhibiting the growth of bacteria or fungus or killing the organisms.

“Targeting” is defined as a technique that amplifies a defined region in nucleic acid.

“Detection” is defined as fluorescent signal that surpasses the background of the threshold.

“Inconclusive” is defined as a result that is valid, but the interpretation does not yield a definitive answer.

“Indeterminate” means not definitely or precisely determined or fixed; not leading to a definite end or result. Examples for indeterminate results might result from inhibitors.

“Diagnosis” is defined as identifying whether a person has bacterial vaginosis (BV), aerobic vaginosis (AV), vulvovaginal candidiasis (VVC), parasitic infections, wellness testing, and/or sexually transmitted infections (STI).

“Wellness testing” indicates the detection of Lactobacillus with or without the detection of other infectious agents, linked to relative sensitivity and specificity.

TABLE 1
Wellness Testing with Calculations based on Detection of Microorganisms

| | | | | | | | True | | False | |
|---|---|---|---|---|---|---|---|---|---|---|
| | | | | | | False | Negatives | True | Negatives | |
| | | | | | | Positives | Not | Positives | 0-3 | |
| | | | | | | in 7-10 | Detected | Detected | Not | Total |
| Analyte | Sensitivity | Specificity | PPV | NPV | Accuracy | Detected | 7-10 | 0-3 | detected | Analyzed |
| Lactobacillus crispatus | 61% | 74% | 76% | 59% | 67% | 34 | 98 | 105 | 67 | 302 |
| Lactobacillus gasseri | 47% | 88% | 83% | 56% | 65% | 16 | 114 | 80 | 90 | 300 |
| Lactobacillus jensenii | 61% | 69% | 71% | 59% | 65% | 40 | 90 | 98 | 62 | 290 |
| All Lactobacillus sp | 60% | 92% | 21% | 80% | 80% | 6 | 68 | 25 | 17 | 115 |
| All Lactobacillus exclude | 82% | 92% | 75% | 94% | 90% | 3 | 34 | 9 | 2 | 48 |
| positive Candida | | | | | | | | | | |
| albicans, C. glabrata | | | | | | | | | | |
| (mixed infections) | | | | | | | | | | |
| All Lactobacillus exclude | 75% | 94% | 75% | 94% | 90% | 2 | 30 | 8 | 2 | 40 |
| positive Candida | | | | | | | | | | |
| albicans, C. glab, C. para | | | | | | | | | | |
| (mixed infections) | | | | | | | | | | |

In practice, a specimen is collected using a solid collection device such as, but not limited to, a flocked swab, brush or broom. The swab is placed against the host's epithelial cell surface and force applied with sweeping motions to collect the sample. The swab is placed in a liquid with preservative properties to stabilize and protect the contents from the swab from degradation. The collected material is allowed to elute from the collecting devices surface and may be vortexed or agitated to encourage the release of materials from the solid collection device's matrix. The contents of the liquid will contain biomarkers from both the host and any infectious entities. The biomarkers are processed for analysis using purification methods. Briefly, an aliquot of the sample is centrifuged to remove the preservative, an extraction control is added to aliquot, and microorganisms are concentrated through salting out procedures. Briefly, a stock solution of 100 ul each of 100% PEG (MW400 W/V), 5M NaCl and extraction control (total 300 ul) is added to 1 ml of clinical specimen, incubated at room temperature for 30 minutes, centrifuged for 2 minutes at 14,000 rpm (16,873×g) and the supernatant removed and the pellet digested using proteinase K. The nucleic acid is extracted or isolated from the sample. Other processes that do not require precipitation, centrifugation and removal of the preservative may be applied.

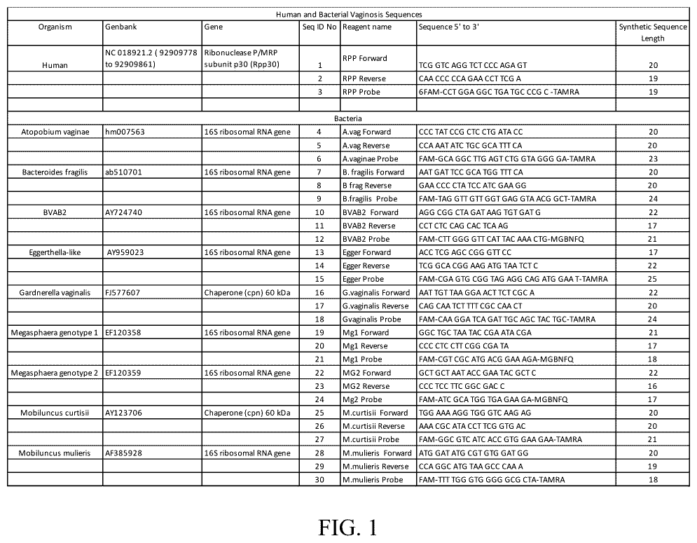

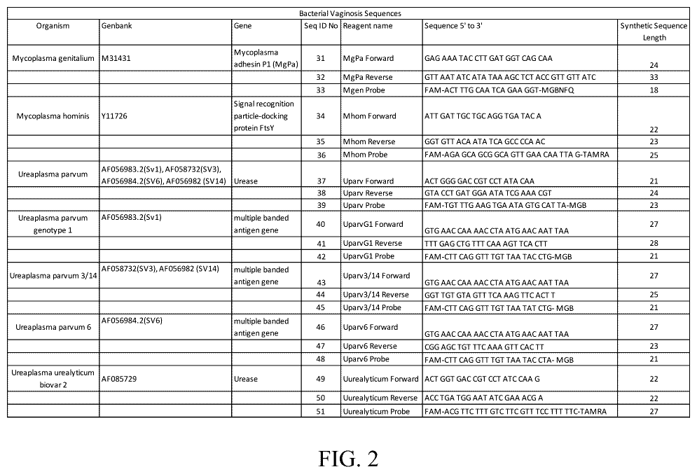

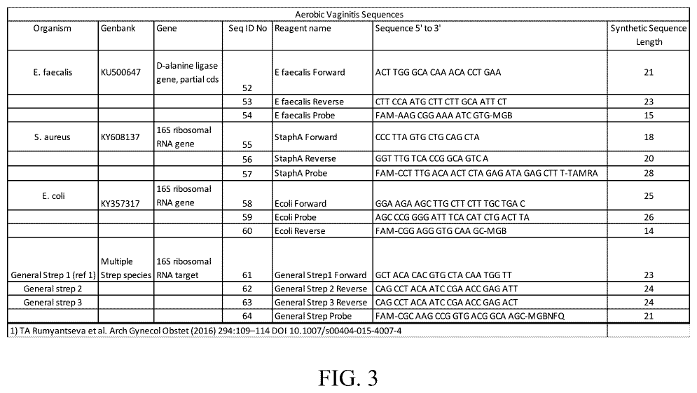

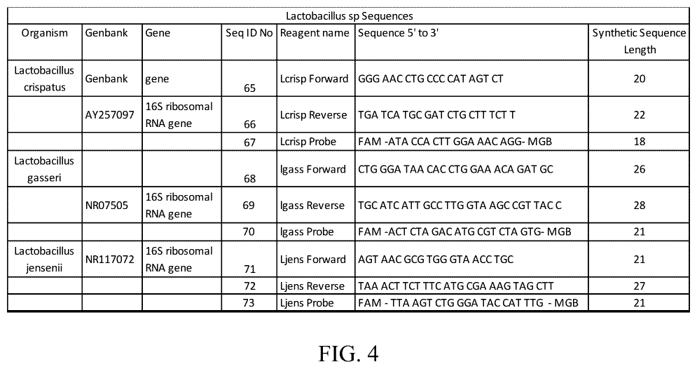

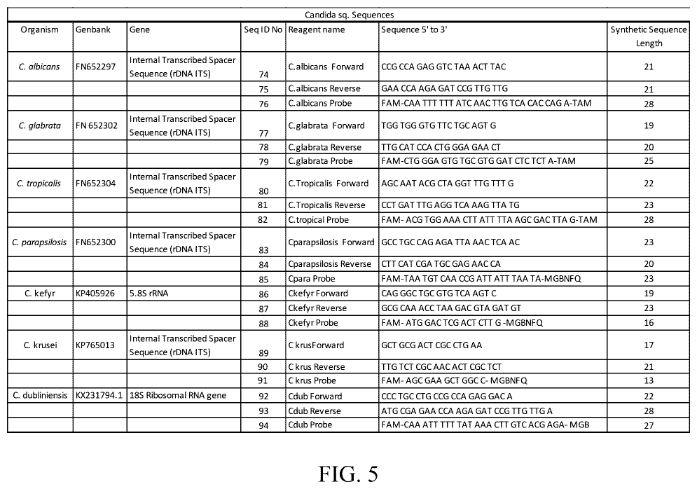

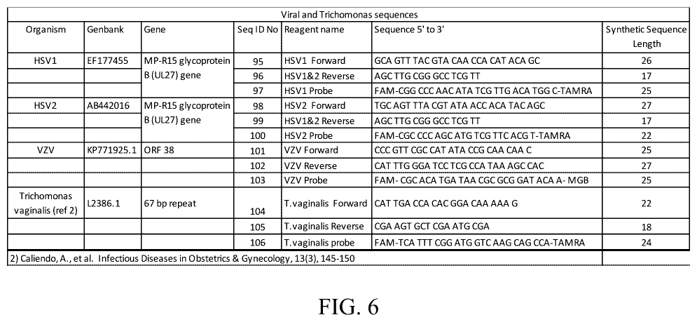

Following purification of the biomarkers, in this case, nucleic acid isolation, the purified nucleic acids are subjected to quantitative polymerase chain reaction (qPCR, q reverse transcriptase PCR, or hybridization) targeting both infectious entities and host cellular contents either simultaneously or in separate reactions. Oligonucleotides in the form of primers and Taqman probes are used to target specific regions of each microbe's genome and the human genome. The fluorescently labeled probe with a quencher molecule is used to detect amplified products. The mixture of primers and probes can be multiplexed or performed separately. The stock concentration (100 μM) of the microorganism's (host's) primers/FAM-labeled probe for each organism. Internal positive control primers and VIC-labeled probe is also added to a master mix to detect inhibition or extraction failure. For a single reaction, the primers/probe mixture is 0.625 μl for each primer, 0.1 μl for each probe, 3.275 of Tris EDTA buffer. The internal positive control is 0.0156 μl for each primer and 0.025 for the probe with 2.4 μl of Tris EDTA. The internal control primer probe mix (2.5 μl per reaction), the microorganism (or host) primer probe mix (3.5 μl), 1 μl of Bovine serum albumin (5 μg/mL) are added to 10 μl of Taqman™ Fast Advanced master mix (catalog number 4444556, ThermoFisher). To this reaction, 3 μl of the extracted sample is added per reaction. The reactions are performed on thermocycler with the following cycling conditions (initial activation at 50° C. for 2 minutes, 90° C. for 20 seconds followed by 39 cycles of 95° C. for 3 sec, 60° C. for 30 S). During the polymerization reaction, the exonuclease activity of the enzyme cleaves the probe, liberating the fluorescent molecule from the quencher molecule. The fluorescent signal is detected in the instrument. The cycle threshold (Ct) is captured by the instrument. Calibrators are included to generate quantitative values. Exemplary primer and probes for the detection of host for the normalization of results for microorganisms and viruses are shown in FIGS. 1-6.

The detection of these biomarkers is quantified using the cycle threshold (Ct) value. The method provides a method for diagnosing bacterial vaginosis (BV), aerobic vaginitis (AV), vulvovaginal candidiasis (VVC), and/or sexually transmitted infections (STI). The amount of the BV, AV, VVC, and/or STI associated infectious biomarker(s) is normalized (i.e., a ratio of the amount of infectious biomarker(s) to the amount of the host biomarker is calculated). The test sample is identified as being positive for BV, AV, VVC, and/or STI associated with one or more infectious biomarkers if the ratio is above a predetermined value. Likewise, the test sample is identified as being negative for a disease associated with the infectious biomarker(s) if the ratio is below the predetermined value. With STI the quantification may not be diagnostic but could be used to measure response to treatment. Many STI such as chlamydia or gonorrhoeae are difficult to culture.

The ratio identifies the detection of the infectious entities at levels consistent with morbidity and provides confidence levels for diagnostic interpretation of results.

In other embodiments, normalizing the first amount of infectious biomarker with the second amount of host biomarker can include comparing the relative infectious and host biomarker amounts with one or more databases of empirical testing data (such as http://www.warfarindosing.org/Source/Home.aspx) which account for both infectious biomarker and host biomarker information (including patient demographic information such as pregnancy status, geographical location, age, menopause, hormone replacement therapy, and transgender status) in various testing samples. Such empirical data can be used to determine which combinations of infectious and host biomarker amounts can correlate to a positive determination for the bacteria in the testing sample. In some instances, a lack of detection of a host biomarker can be accounted for and used to normalize a testing sample. Such databases can also help determine confidence, specificity, sensitivity, and accuracy values for a given testing sample.

In the particular embodiments, individual or multiple assays for specific bacteria for BV such as Atopobium vaginae, Gardnerella vaginalis, bacterial vaginosis associated bacterium 2 (BVAB2), Megaspheara type 1, Mycoplasma hominis, Mobiluncus curtisii, Ureaplasma urealyticum, Ureaplasma parvum (genotypes) and/or Lactobacillus sp are detected along with the host associated biomarker (e.g., RPP30).

Specimen collection using collection devices vary significantly between individuals with some individuals aggressively collecting specimens while other individuals prefer a gentle approach. Aggressive collection results in a high concentration and amount of the host biomarker.

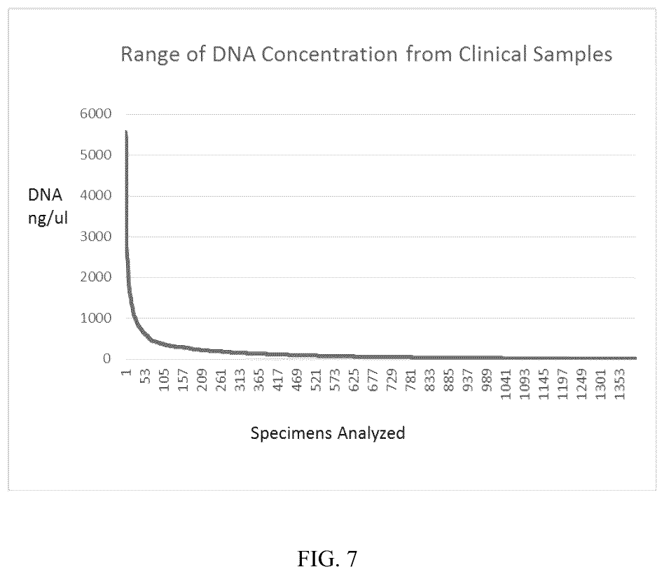

Referring now to FIG. 7, there is shown a range of human biomarkers detected in specimens collected for routine clinical testing. Variability in collection methods results in a range from 0 to more than 5,000 ng/μl of DNA;

Specimens with high amounts of the host biomarker may produce a sample less accurate (specifically, 52% compared with 82%) with low amounts, as illustrated in Table 2, below.

TABLE 2
Diagnostic Value with Host Biomarker Quantitative Value

| | High | Low |
|---|---|---|
| Sensitivity | 62% | 100% |
| Specificity | 44% | 71% |
| PPV | 47% | 67% |
| NPV | 58% | 100% |
| Accuracy | 52% | 82% |

Additional data employing the detection of a noninfectious (aka host) biomarkers to address variability in collection methods improves diagnostic accuracy of methods used to detect infectious agent biomarkers.

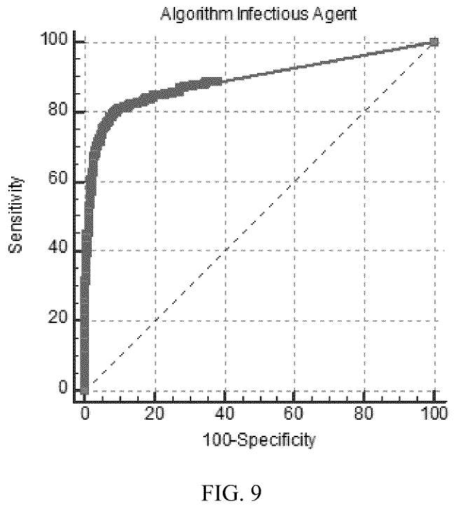

Referring now also to FIG. 9, there is shown the receiver operator curve (RoC) associated with more than 1,000 determinations using only a quantitative biomarker associated with a single infectious agent and a reference method for disease. Note the large gap of information for specificity in the graph with no data points below 65% specificity.

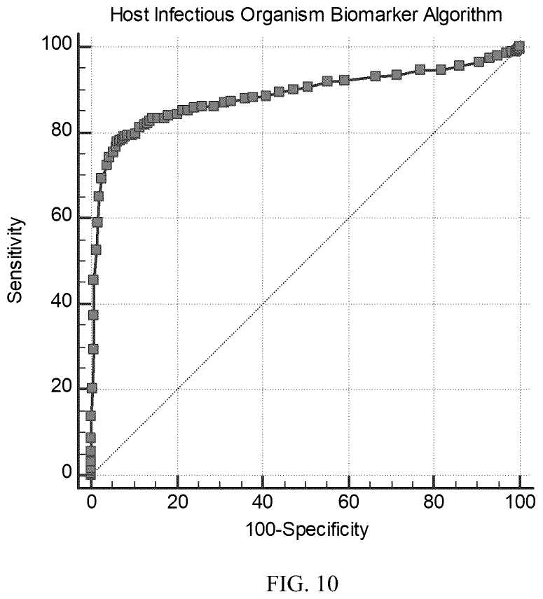

In contrast, FIG. 10 demonstrates a RoC when an algorithm incorporating a quantitative host biomarker is used to normalize data generated from a quantitative biomarker for the infectious agent. Although the ROC illustrates how to use the normalization to generate a specificity number, it should be understood that other RoC curves for vulvovaginal candidiasis and aerobic vaginitis can be used to predict numerical calculations for samples with multiple infectious entities. The gap with interpretative value is eliminated with incorporating the host biomarker in the algorithm for assessing the diagnostic value of the test. RoC analysis was performed using the quantitative analysis of the host biomarker without inclusion of the infectious biomarker data and no correlation with the host biomarker alone with the disease state was identified.

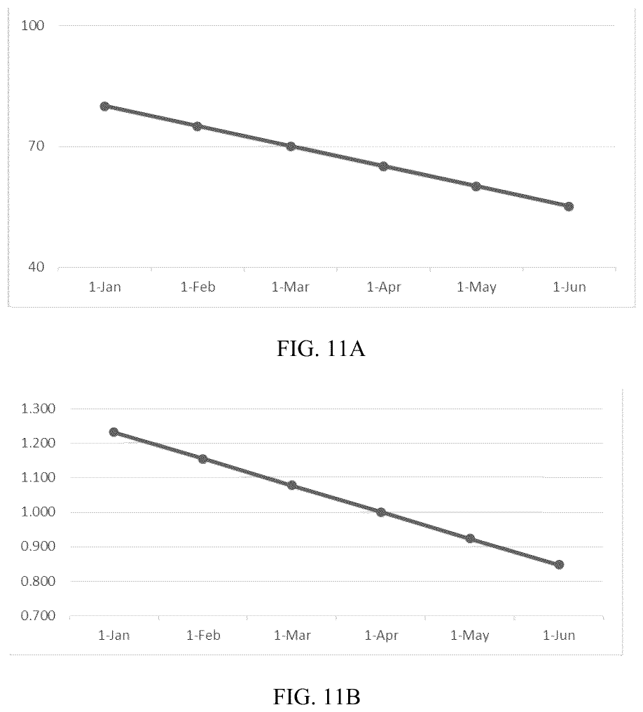

Referring now to FIGS. 11-14, examples are provided of various interpretations of results with and without normalizing the detection of the infectious agent against the host biomarker. If the collection of host biomarker were constant, the results demonstrate consistent results with and without normalization (FIG. 11). However, as is demonstrated in FIG. 7, collection amounts differ.

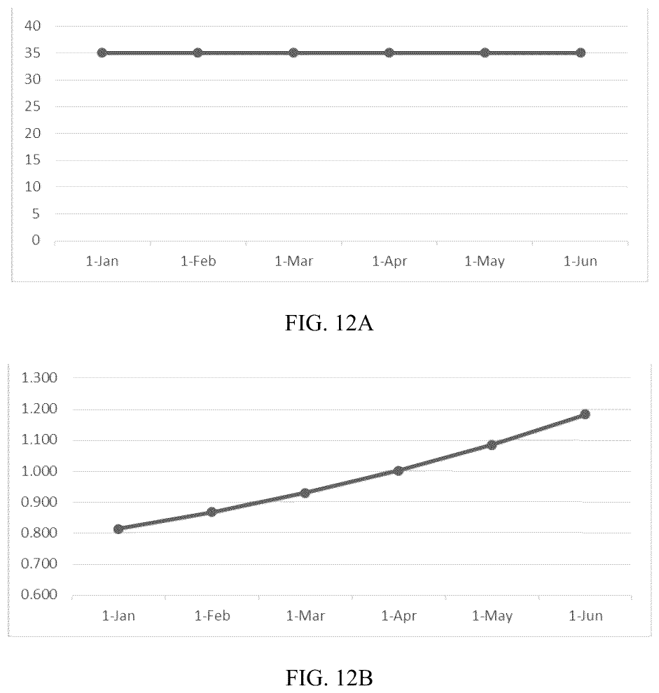

Referring now to FIGS. 12a and 12b, the potential for inaccurate interpretation without normalization is illustrated whereby a provider may consider the patient to have a stable level of infectious agents when the normalized values demonstrate a change of increasing infectious agent load. FIG. 12b illustrates the inclusion of a host biomarker data algorithm, indicating an increase in the relative amount of infectious agent, which may indicate increasing levels of morbidity or failure to respond to treatment.

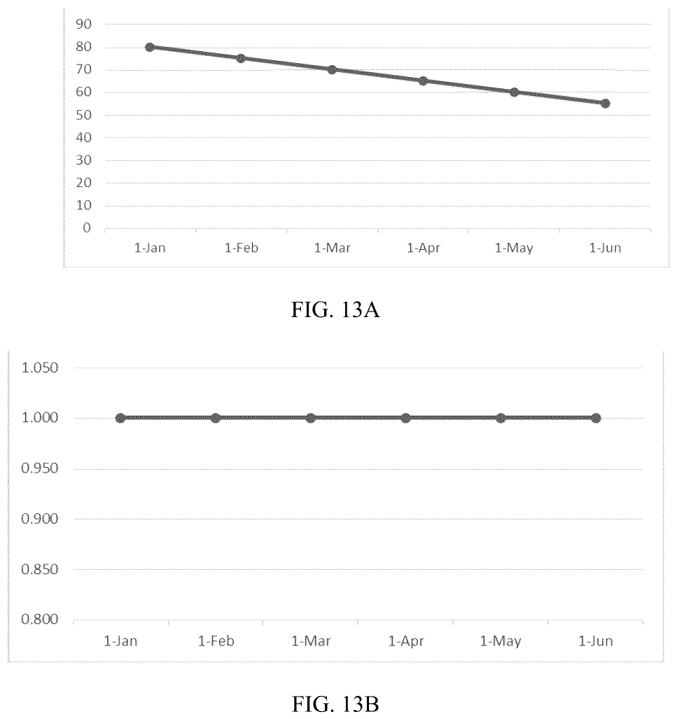

FIGS. 13a and 13b illustrate another potential misinterpretation where non-normalized data appear to represent an improvement or decrease in infectious agent that suggests a return to the normal microbiota when the normalized data indicate status. FIG. 13b is a graph similar to that shown in FIG. 13a, following normalization with a biomarker data algorithm, the results demonstrating a static state for an infectious agent.

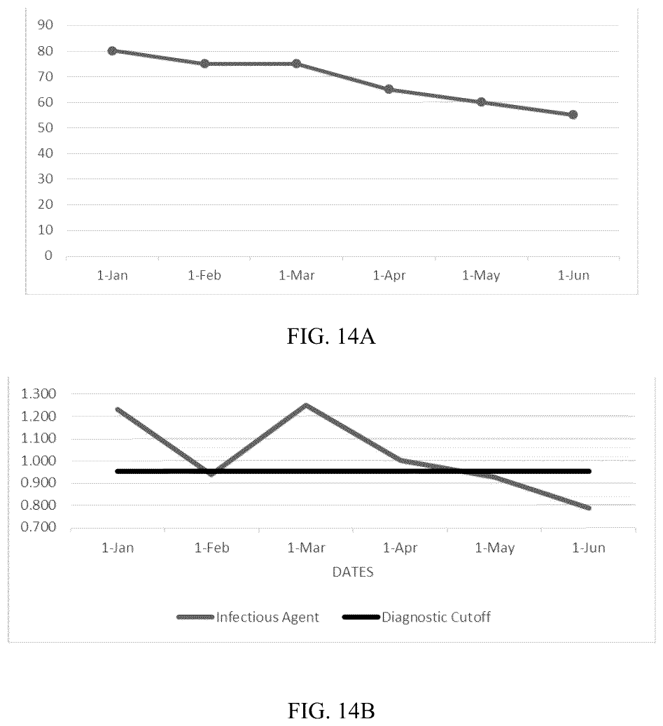

FIGS. 14a and 14b illustrate non-normalized data that appear to support a downward trend of infectious agent when the normalized data illustrate variability in infectious agent quantity. FIG. 14b is a graph similar to that shown in FIG. 14a, depicting decreasing levels of an infectious agent over time with the data normalized with a host biomarker algorithm and significant variability occurs. Inclusion of a “diagnostic cutoff line” may guide clinical decisions as to whether additional treatment is necessary. Algorithm values below the diagnostic cutoff would be consider normal results and would not require treatment. A diagnostic cutoff or reference line can be applied that may inform the healthcare provider that values associated with disease (above the diagnostic cutoff or reference line) compared with non-disease states (i.e., values below the diagnostic cutoff or reference line)

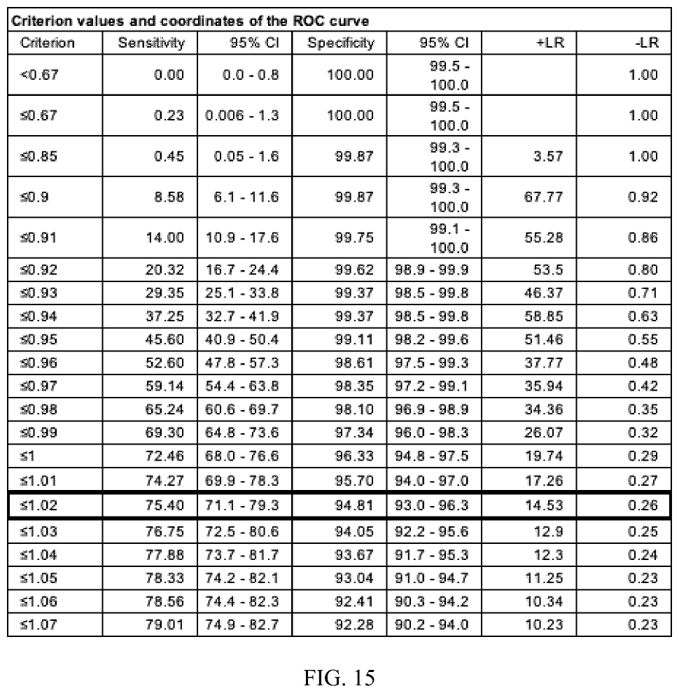

FIG. 15 is a partial table derived from a database showing sensitivity with specificity after applying an algorithm, which information may be used to set reference lines for diagnostic interpretation. This example shows how database and diagnostic values can be customized to analyte or disease population. Specifically, this example demonstrates how the algorithm setting of 1.02 can be used to obtain a sensitivity of 75.4 and a specificity of 94.81.

In one embodiment, the cycle threshold value was applied to one or more bacteria associated with bacterial vaginosis such as Atopobium vaginae, Gardnerella vaginalis, bacterial vaginosis associated bacterium 2 (BVAB2), Megaspheara type 1. Mycoplasma hominis, Mobiluncus curtisii, Ureaplasma parvum (genotypes) and Ureaplasma urealyticum and/or Lactobacillus sp. Infectious agent biomarker Ct values greater than a diagnostic cutoff (e.g., 39 Ct) with results of host biomarker (RPP30) greater than the diagnostic cutoff is considered an inadequate sample. This result is reported as indeterminate or inconclusive.

In another embodiment, the cycle threshold value was applied to one or more bacteria associated with aerobic vaginitis such as Escherichia coli, Group B Streptococcus and other Streptococcus spp., Staphylococcus aureus, and Enterococcus faecalis and/or Lactobacillus sp. Infectious agent biomarker Ct values greater than diagnostic cutoff (e.g., 39 Ct) with results of host biomarker (RPP30) is greater than diagnostic cutoff is considered an inadequate sample. This result is reported as indeterminate or inconclusive.

In another embodiment, the cycle threshold value was applied to one or more yeast associated with vulvovaginal candidiasis (VVC) such as Candida albicans and/or Candida glabrata and/or Lactobacillus sp. Infectious agent biomarker Ct values of greater than a diagnostic cutoff (e.g., 39 Ct) with results of host biomarker (RPP30) is greater than the diagnostic cutoff is considered an inadequate sample. This result is also reported as indeterminate or inconclusive.

In another embodiment, the cycle threshold value was applied to one or more bacteria associated with bacterial vaginosis such as Atopobium vaginae, Gardnerella vaginalis, bacterial vaginosis associated bacterium 2 (BVAB2), Megaspheara type 1, Mycoplasma hominis, Mobiluncus curtisii, Ureaplasma parvum (genotypes) and Ureaplasma urealyticum and/or Lactobacillus sp. Infectious agent biomarker Ct values of less than a diagnostic cutoff (e.g., 39 Ct) with results of host biomarker (RPP30) is less than the diagnostic cutoff is considered an adequate sample and is considered detected or positive.

In another embodiment, the cycle threshold value was applied to one or more bacteria associated with aerobic vaginitis such as Escherichia coli, Group B Streptococcus and other Streptococcus spp., Staphylococcus aureus, and Enterococcus faecalis and/or Lactobacillus sp. Infectious agent biomarker Ct values less than a diagnostic cutoff (e.g., 39 Ct) with results of host biomarker (RPP30) is less than the diagnostic cutoff is considered an adequate sample and considered detected or positive.

In another embodiment, the cycle threshold value was applied to one or more yeast associated with vulvovaginal candidiasis (VVC) such as Candida albicans and/or Candida glabrata and/or Lactobacillus sp. Infectious agent biomarker Ct values of less than a diagnostic cutoff (e.g., 39 Ct) with results of host biomarker (RPP30) is less than the diagnostic cutoff is considered an adequate sample and considered detected or positive.

In another embodiment, the cycle threshold value was applied to one or more bacteria associated with bacterial vaginosis such as Atopobium vaginae, Gardnerella vaginalis, bacterial vaginosis associated bacterium 2 (BVAB2), Megaspheara type 1, Mycoplasma hominis, Mobiluncus curtisii, Ureaplasma parvum (genotypes) and Ureaplasma urealyticum and/or Lactobacillus sp. Infectious agent biomarker Ct values of less than a diagnostic cutoff (e.g., 39 Ct) with results of host biomarker (RPP30) is greater than the diagnostic cutoff is considered an adequate sample and is considered detected or positive.

In another embodiment, the cycle threshold value was applied to one or more bacteria associated with aerobic vaginitis such as Escherichia coli, Group B Streptococcus and other Streptococcus spp., Staphylococcus aureus, and Enterococcus faecalis and/or Lactobacillus sp. Infectious agent biomarker Ct values less than a diagnostic cutoff (e.g., 39 Ct) with results of host biomarker (RPP) is greater than the diagnostic cutoff is considered an adequate sample and considered detected or positive.

In another embodiment, the cycle threshold value was applied to one or more yeast associated with vulvovaginal candidiasis (VVC) such as Candida albicans and/or Candida glabrata and/or Lactobacillus sp. Infectious agent biomarker Ct values of less than a diagnostic cutoff (e.g., 39 Ct) with results of host biomarker (RPP30) is greater than the diagnostic cutoff is considered an adequate sample and considered detected or positive.

In another embodiment, the cycle threshold value was applied to one or more bacteria associated with bacterial vaginosis such as Atopobium vaginae, Gardnerella vaginalis, bacterial vaginosis associated bacterium 2 (BVAB2), Megaspheara type 1, Mycoplasma hominis, Mobiluncus curtisii, Ureaplasma parvum (genotypes) and Ureaplasma urealyticum and/or Lactobacillus sp. Infectious agent biomarker Ct values of less than a diagnostic cutoff (e.g., 39 Ct) with results of host biomarker (RPP30) is less than the diagnostic cutoff is considered an adequate sample and is considered positive. The results of specimens with multiple positive organisms have associated specificity, sensitivity, and accuracy scores associated with the lab result so to guide diagnosis with confidence.

In another embodiment, the Ct value was applied to one or more bacteria associated with aerobic vaginitis such as Escherichia coli, Group B Streptococcus and other Streptococcus spp., Staphylococcus aureus, and Enterococcus faecalis and/or Lactobacillus sp. Infectious agent biomarker Ct values of less than a diagnostic cutoff (e.g., 39 Ct) with results of host biomarker (RPP30) is less than the diagnostic cutoff is considered an adequate sample and is considered positive. The results of specimens with multiple positive organisms have associated specificity, sensitivity, and accuracy scores associated with the lab result so to guide diagnosis with confidence.

In another embodiment, the cycle threshold value was applied to one or more bacteria associated with bacterial vaginosis such as Atopobium vaginae, Gardnerella vaginalis, bacterial vaginosis associated bacterium 2 (BVAB2), Megaspheara type 1, Mycoplasma hominis, Mobiluncus curtisii, Ureaplasma parvum (genotypes) and/or Ureaplasma urealyticum and/or Lactobacillus sp, where the following algorithm for the host to infectious agent biomarker is calculated:

$$\frac{100 - Host Marker ( Ct )}{100 - infectious agent biomarker Ct values}$$

$$\frac{100 - Host Marker ( Ct )}{{Ave ( 100 - x , 100 - y , 100 - z , … )}^{*}}$$

The reference range is organism specific and linked to database values identifying confidence levels for specificity, sensitivity, and accuracy of results with diagnosing BV. Results below a threshold (e.g., 50% specificity) were reported as indeterminate or inconclusive. *when infectious agent is detected at a given threshold

In one embodiment, the cycle threshold (Ct) value is applied to one or more bacteria associated with aerobic vaginitis such as Escherichia coli, Group B Streptococcus and other Streptococcus spp., Staphylococcus aureus, and Enterococcus faecalis and/or Lactobacillus sp, where the following algorithm for the host to infectious agent biomarker is calculated:

$$\frac{100 - Host Marker ( Ct )}{100 - infectious agent biomarker Ct values}$$

$$\frac{100 - Host Marker ( Ct )}{{Ave ( 100 - x , 100 - y , 100 - z , … )}^{*}}$$

The reference range is organism specific and linked to database values identifying confidence levels for specificity, sensitivity, and accuracy of results with diagnosing AV. Results below a threshold (e.g., 50% specificity) were reported as indeterminate or inconclusive. *when infectious agent is detected at a given threshold

In another embodiment, the cycle threshold (Ct) value was applied to one or more yeasts associated with VVC such as Candida albicans and Candida glabrata and/or Lactobacillus sp. Where the following ratio for the host to infectious agent biomarker is calculated:

$$\frac{100 - Host Biomarker ( Ct )}{100 - infectious agent biomarker Ct values}$$

$$\frac{100 - Host Marker ( Ct )}{{Ave ( 100 - x , 100 - y , 100 - z , … )}^{*}}$$

The reference range is organism specific and linked to database values identifying confidence levels for specificity, sensitivity, and accuracy of results with diagnosing VVC. Results below a threshold (e.g., 50% specificity) were reported as indeterminate or inconclusive. *when infectious agent is detected at a given threshold

In another embodiment, the specificity, sensitivity, and accuracy scores were included in the laboratory result for BV associated infectious agents providing personalized medicine reporting.

In another embodiment, the ratio/algorithm value for BV was displayed either in a graph or text for each patient providing longitudinal data and tracking of laboratory results over time. This display provided normalized data to track treatment response or failure. (See examples in FIGS. 11-14).

In another embodiment, the specificity, sensitivity and accuracy scores were included in the laboratory result for AV associated infectious agents providing personalized medicine reporting.

In another embodiment, the ratio/algorithm value for AV associated biomarkers was displayed either in a graph or text for each patient providing longitudinal data and tracking of laboratory results over time. This display provides normalized data to track treatment response or failure.

In another embodiment, the specificity, sensitivity and accuracy scores were included in the laboratory result for VVC associated infectious agents providing personalized medicine reporting.

In another embodiment, the ratio/algorithm value for VVC associated biomarkers was displayed either in a graph or text for each patient providing longitudinal data and tracking of laboratory results over time. This display provides normalized data to track treatment response or failure.

In another embodiment, where various sexually transmitted infectious agents (e.g., CT/NG, Trichomonas vaginalis, Mycoplasma genitalium) is not detected (e.g., using IVDs tests or other testing methods) have the sample also tested for the host biomarker (e.g., RPP30) was greater than diagnostic cutoff (e.g., 39 Ct) is considered an inadequate sample. This result was reported as indeterminate or inconclusive.

Addition of wellness score incorporating the detection of Lactobacillus sp can further refine the normalization of the data. It should be understood that the ratio and algorithms including the host biomarker described herein are intended as examples only.

In some embodiments, the results of a testing sample can be delivered with a confidence rating (i.e., low positive, medium positive, high positive, low negative, medium negative, high negative, etc.). The confidence rating provides insight to the physician or review of the testing results as to the relative confidence or certainty of the diagnosis. The confidence rating can also be used as a specificity or sensitivity rating.

In some embodiments, the confidence ratings can be based on the ratios of the quantities of the infectious bacteria biomarker to the host biomarker determined from the algorithm. For instance, if the threshold for a positive diagnosis is a ratio of 0.5 or higher, ranges above such a ratio, for instance 0.5-0.7, 0.7-1.0, and >1.0 can be associated with a confidence rating of a low, medium, and high positive, respectively. This approach can be part of the specificity score. In cases where the patient is pregnant, the score may be shifted to be more sensitive or less specific due to the potential harm to pregnancy.

In other embodiments, the confidence rating determination can also take into account actual quantity of a host biomarker and/or infectious biomarker. If a positive or negative diagnosis is achieved, then a physician would likely have more confidence in the diagnosis if the sample size was larger vs if the sample size was smaller. For instance, in the example above, if the calculated ratio in a sample was 0.6, it would be assigned a low positive confidence threshold based on the ratio alone. However, if the physical quantity of host biomarker and/or infectious biomarker were determined to be a large amount, indicative of a large sample size, that determination of a large sample size may affect the confidence rating, such that the confidence rating could be increased to a medium confidence given the larger sample size. Similar analysis between the determined ratios and quantitative values for the host and infectious bacteria biomarkers can be provided for the entire spectrum of potential results and ratio/quantitative data combinations (FIG. 14B). Such confidence thresholds for the different combinations of ratio/quantitative data combinations can be ascertained from empirical data.

In other embodiments, the confidence and various other statistical interpretations can be based on empiric data from a database.

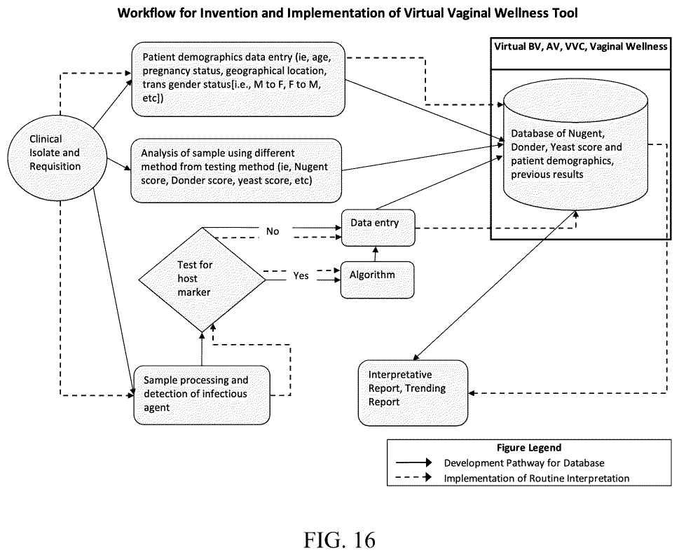

FIG. 16 illustrates the development and utilization of information for a virtual vaginal wellness tool, whereby empiric data is captured along with demographic information for samples presenting with similar results.

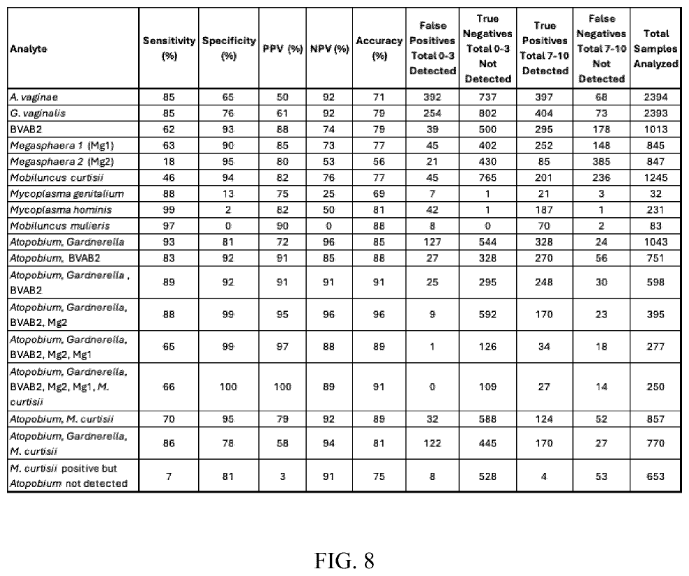

FIG. 8 illustrates various diagnostic values associated with specimens containing multiple organisms. Examples of results that would be pulled from virtual database of tested samples. All fields may not be shown in the result for the healthcare provider.

Thus, although there have been described particular embodiments of the present invention of a new and useful host/infectious disease biomarker testing, it is not intended that such references be construed as limitations upon the scope of this invention. All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

It should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a great number of variations of the devices, device components, and method steps set forth in the present description. As will be obvious to one of skill in the art, methods and devices useful for the present methods can include a great number of optional composition and processing elements and steps.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are claimed, it should be understood that compounds known and available in the art prior to Applicant's invention, including compounds for which an enabling disclosure is provided in the references cited herein, are not intended to be included in any composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of,” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A method of testing for the presence of an infectious biomarker by using a database to algorithmically normalize the results comprising:
a. collecting a test sample from a patient comprising of at least one infectious biomarker and a second amount of a host biomarker;
b. determining the first amount of at least one infectious biomarker in the test sample;
c. determining the second amount of the host biomarker in the test sample; and
d. utilizing a database to algorithmically normalize the first amount of at least one infectious biomarker as a function of the second amount of the host biomarker.

2. The method of testing for the presence of an infectious biomarker in accordance with claim 1, wherein there is an identification of the test sample as positive or negative that is determined by a database which produces RoC curves utilizing a variety of data inputs such as patient demographics clinical presentations and wellness.

3. The method of testing for the presence of an infectious biomarker in accordance with claim 2, the steps further comprising:
a. graphically displaying the algorithmically normalized results over a course of time to monitor for signs of clinical relapse or for success of the initially prescribed cure.

4. A method of testing for the presence of an infectious biomarker by using a database to algorithmically normalize the results comprising:
a. collecting a test sample from a patient comprising of at least one infectious biomarker and a second amount of a host biomarker;
b. determining the first amount of at least one infectious biomarker in the test sample;
c. determining the second amount of the host biomarker in the test sample;
d. utilizing a database to normalize the first amount of at least one infectious biomarker as a function of the second amount of the host biomarker; and
e. identifying the test sample as positive for a disease associated with at least one infectious biomarker if the ratio of the first amount of infectious biomarker to the second amount of the host biomarker is above a predetermined value.

5. The method of testing for the presence of an infectious biomarker in accordance with claim 4, wherein the sensitivity and specificity are determined by an RoC curve using statistical methods.

6. The method of testing for the presence of an infectious biomarker in accordance with claim 5, wherein the cycle threshold value for the ROC curve is determined using the algorithms as defined in the specification.

7. The method of testing for the presence of an infectious biomarker in accordance with claim 4, wherein the normalizing step 4(e) is performed with an algorithm in the form of one of a set of formulae consisting of:
$$a . \frac{host biomarker ( s )}{infectious biomarker ( s )} and \frac{infectious agent biomarker ( s )}{host biomarker ( s ) .}$$

8. The method of testing for the presence of an infectious biomarker in accordance with claim 5, wherein the database comprises a set of at least one patient factor consisting essentially of: demographic information, infectious disease, presence of beneficial commensal organisms, prior infections, non-human pathogens such as bacteriophages, gender, age, biographical ancestry, pregnancy status, treatment, location, zip code, sexual activity, transgender status, number of different sexual partners over a predetermined period of time, sexual preference, sexual practices, smoking status, hormone replacement recipient, menopause, dental dams, contraceptive use, type of condoms or wellness.

9. The method of testing for the presence of an infectious biomarker in accordance with claim 5, the steps further comprising:
a. graphically displaying the normalized results over a course of time to monitor for signs of clinical relapse or for success of the initially prescribed cure or wellness.

10. A method of testing for the presence of vaginal colonizations or wellness by using a database to algorithmically normalize the results comprising:
a. collecting a test sample from a patient containing at least one biomarker of a vaginal colonization and a second amount of a host biomarker;
b. determining the first amount of at least one biomarker of a vaginal colonization in the test sample;
c. determining the second amount of the host biomarker in the test sample;
d. creating normalized results by normalizing the first amount of at least one biomarker of a vaginal colonization as a function of the second amount of the host biomarker by utilizing a database containing patient demographics; and
e. utilizing the normalized results to determine a positive result for a disease or wellness associated with at least one biomarker of a vaginal colonization if the ratio of the first amount of the biomarker of a vaginal colonization to the second amount of the host biomarker is above a predetermined value.

11. The method of testing for the presence of vaginal colonization in accordance with claim 10, further comprising using a database to algorithmically normalize the results, whereby the normalization of results is performed by algorithms which create individualized RoC curves by factoring in patient demographics using statistical methods.

12. The method of testing for the presence of vaginal colonization by using a database to algorithmically normalize the results in accordance with claim 11, wherein the database comprises a set of at least one of the following patient demographic data: demographic information, infectious disease, presence of beneficial commensal organisms, prior infections, non-human pathogens such as bacteriophages, gender, age, biogeographical ancestry, pregnancy status, treatment, location, zip code, sexual activity, transgender status, number of different sexual partners over a predetermined period of time, sexual preference, sexual practices, smoking status, hormone replacement recipient, menopause, dental dams, contraceptive use, or type of condoms or wellness.

13. The method of testing for the presence of vaginal colonizations by using a database to algorithmically normalize the results in accordance with claim 11, the steps further comprising:
a. graphically displaying the normalized results over a course of time to monitor for signs of clinical relapse or for success or wellness of the initially prescribed cure.

14. The method of testing for the presence of vaginal colonization by using a database to algorithmically normalize the results in accordance with claim 11, the steps further comprising:
a. providing recommendations for post treatment such as an extension of the course of treatment or an increase in treatment where monitoring has shown signs of clinical relapse.


SEQUENCE LISTING (94 sequences)

SEQ ID NO: 1 - DNA, 20 bp, Homo sapiens
  source 1..20: mol_type = other DNA
tcggtcaggtctcccagagt

SEQ ID NO: 2 - DNA, 19 bp, Homo sapiens
  source 1..19: mol_type = other DNA
caacccccagaaccttcga

SEQ ID NO: 3 - DNA, 19 bp, Homo sapiens
  source 1..19: mol_type = other DNA
cctggaggctgatgcccgc

SEQ ID NO: 4 - DNA, 20 bp, Atopobium vaginae
  source 1..20: mol_type = other DNA
ccctatccgctcctgatacc

SEQ ID NO: 5 - DNA, 20 bp, Atopobium vaginae
  source 1..20: mol_type = other DNA
ccaaatatctgcgcatttca

SEQ ID NO: 6 - DNA, 23 bp, Atopobium vaginae
  source 1..23: mol_type = other DNA
gcaggcttgagtctggtagggga

SEQ ID NO: 7 - DNA, 20 bp, Bacteroides fragilis
  source 1..20: mol_type = other DNA
aatgattccgcatggtttca

SEQ ID NO: 8 - DNA, 20 bp, Bacteroides fragilis
  source 1..20: mol_type = other DNA
gaacccctatccatcgaagg

SEQ ID NO: 9 - DNA, 24 bp, Bacteroides fragilis
  source 1..24: mol_type = other DNA
taggttgttggtgaggtaacggct

SEQ ID NO: 10 - DNA, 22 bp, Oscillospira sp.
  source 1..22: mol_type = other DNA
aggcggctagataagtgtgatg

SEQ ID NO: 11 - DNA, 17 bp, Oscillospira sp.
  source 1..17: mol_type = other DNA
cctctccagcactcaag

SEQ ID NO: 12 - DNA, 21 bp, Oscillospira sp.
  source 1..21: mol_type = other DNA
cttggggttcattacaaactg

SEQ ID NO: 13 - DNA, 17 bp, Eggerthella sp.
  source 1..17: mol_type = other DNA
acctcgagccgggttcc

SEQ ID NO: 14 - DNA, 22 bp, Eggerthella sp.
  source 1..22: mol_type = other DNA
tcggcacggaagatgtaatctc

SEQ ID NO: 15 - DNA, 25 bp, Eggerthella sp.
  source 1..25: mol_type = other DNA
cgagtgcggtagaggcagatggaat

SEQ ID NO: 16 - DNA, 22 bp, Gardnerella vaginalis
  source 1..22: mol_type = other DNA
aattgttaaggaacttctcgca

SEQ ID NO: 17 - DNA, 20 bp, Gardnerella vaginalis
  source 1..20: mol_type = other DNA
cagcaatcttttcgccaact

SEQ ID NO: 18 - DNA, 24 bp, Gardnerella vaginalis
  source 1..24: mol_type = other DNA
caaggatcagattgcagctactgc

SEQ ID NO: 19 - DNA, 21 bp, Megasphaera lornae
  source 1..21: mol_type = other DNA
ggctgctaataccgaatacga

SEQ ID NO: 20 - DNA, 17 bp, Megasphaera lornae
  source 1..17: mol_type = other DNA
cccctccttcggcgata

SEQ ID NO: 21 - DNA, 18 bp, Megasphaera lornae
  source 1..18: mol_type = other DNA
cgtcgcatgacggaaaga

SEQ ID NO: 22 - DNA, 22 bp, Megasphaera hutchinsoni
  source 1..22: mol_type = other DNA
gctgctaataccgaatacgctc

SEQ ID NO: 23 - DNA, 16 bp, Megasphaera hutchinsoni
  source 1..16: mol_type = other DNA
ccctccttcggcgacc

SEQ ID NO: 24 - DNA, 17 bp, Megasphaera hutchinsoni
  source 1..17: mol_type = other DNA
atcgcatggtgagaaga

SEQ ID NO: 25 - DNA, 20 bp, Mobiluncus curtisii
  source 1..20: mol_type = other DNA
tggaaaaggtgggtcaagag

SEQ ID NO: 26 - DNA, 20 bp, Mobiluncus curtisii
  source 1..20: mol_type = other DNA
aaacgcataccttcggtgac

SEQ ID NO: 27 - DNA, 21 bp, Mobiluncus curtisii
  source 1..21: mol_type = other DNA
ggcgtcatcaccgtggaagaa

SEQ ID NO: 28 - DNA, 20 bp, Mobiluncus mulieris
  source 1..20: mol_type = other DNA
atggatatgcgtgtggatgg

SEQ ID NO: 29 - DNA, 19 bp, Mobiluncus mulieris
  source 1..19: mol_type = other DNA
ccaggcatgtaagcccaaa

SEQ ID NO: 30 - DNA, 18 bp, Mobiluncus mulieris
  source 1..18: mol_type = other DNA
ttttgggtgggggcgcta

SEQ ID NO: 31 - DNA, 24 bp, Mycoplasma genitalium
  source 1..24: mol_type = other DNA
gagaaataccttgatggtcagcaa

SEQ ID NO: 32 - DNA, 33 bp, Mycoplasma genitalium
  source 1..33: mol_type = other DNA
gttaatatcatataaagctctaccgttgttatc

SEQ ID NO: 33 - DNA, 18 bp, Mycoplasma genitalium
  source 1..18: mol_type = other DNA
actttgcaatcagaaggt

SEQ ID NO: 34 - DNA, 22 bp, Mycoplasma hominis
  source 1..22: mol_type = other DNA
attgattgctgcaggtgataca

SEQ ID NO: 35 - DNA, 23 bp, Mycoplasma hominis
  source 1..23: mol_type = other DNA
ggtgttacaatatcagccccaac

SEQ ID NO: 36 - DNA, 25 bp, Mycoplasma hominis
  source 1..25: mol_type = other DNA
agagcagcggcagttgaacaattag

SEQ ID NO: 37 - DNA, 21 bp, Ureaplasma parvum
  source 1..21: mol_type = other DNA
actggggaccgtcctatacaa

SEQ ID NO: 38 - DNA, 24 bp, Ureaplasma parvum
  source 1..24: mol_type = other DNA
gtacctgatggaatatcgaaacgt

SEQ ID NO: 39 - DNA, 23 bp, Ureaplasma parvum
  source 1..23: mol_type = other DNA
tgtttgaagtgaatagtgcatta

SEQ ID NO: 40 - DNA, 27 bp, Ureaplasma parvum
  source 1..27: mol_type = other DNA
gtgaaccaaaacctaatgaacaattaa

SEQ ID NO: 41 - DNA, 24 bp, Ureaplasma parvum
  source 1..24: mol_type = other DNA
tttgagctgtttcaaagttcactt

SEQ ID NO: 42 - DNA, 21 bp, Ureaplasma parvum
  source 1..21: mol_type = other DNA
cttcaggtttgttaatacctg

SEQ ID NO: 43 - DNA, 27 bp, Ureaplasma parvum
  source 1..27: mol_type = other DNA
gtgaaccaaaacctaatgaacaattaa

SEQ ID NO: 44 - DNA, 25 bp, Ureaplasma parvum
  source 1..25: mol_type = other DNA
ggttgtgtagtttcaaagttcactt

SEQ ID NO: 45 - DNA, 21 bp, Ureaplasma parvum
  source 1..21: mol_type = other DNA
cttcaggtttgttaatatctg

SEQ ID NO: 46 - DNA, 27 bp, Ureaplasma parvum
  source 1..27: mol_type = other DNA
gtgaaccaaaacctaatgaacaattaa

SEQ ID NO: 47 - DNA, 23 bp, Ureaplasma parvum
  source 1..23: mol_type = other DNA
cggagctgtttcaaagttcactt

SEQ ID NO: 48 - DNA, 21 bp, Ureaplasma parvum
  source 1..21: mol_type = other DNA
cttcaggtttgttaataccta

SEQ ID NO: 49 - DNA, 22 bp, Ureaplasma parvum
  source 1..22: mol_type = other DNA
actggtgaccgtcctatccaag

SEQ ID NO: 50 - DNA, 22 bp, Ureaplasma parvum
  source 1..22: mol_type = other DNA
acctgatggaatatcgaaacga

SEQ ID NO: 51 - DNA, 27 bp, Ureaplasma parvum
  source 1..27: mol_type = other DNA
acgttctttgtcttcgtttcctttttc

SEQ ID NO: 52 - DNA, 21 bp, Enterococcus faecalis
  source 1..21: mol_type = other DNA
acttgggcacaaacacctgaa

SEQ ID NO: 53 - DNA, 23 bp, Enterococcus faecalis
  source 1..23: mol_type = other DNA
cttccaatgcttcttgcaattct

SEQ ID NO: 54 - DNA, 15 bp, Enterococcus faecalis
  source 1..15: mol_type = other DNA
aagcggaaaatcgtg

SEQ ID NO: 55 - DNA, 18 bp, Staphylococcus aureus
  source 1..18: mol_type = other DNA
cccttagtgctgcagcta

SEQ ID NO: 56 - DNA, 19 bp, Staphylococcus aureus
  source 1..19: mol_type = other DNA
ggtttgtcaccggcagtca

SEQ ID NO: 57 - DNA, 28 bp, Staphylococcus aureus
  source 1..28: mol_type = other DNA
cctttgacaactctagagatagagcttt

SEQ ID NO: 58 - DNA, 25 bp, Escherichia coli
  source 1..25: mol_type = other DNA
ggaagaagcttgcttctttgctgac

SEQ ID NO: 59 - DNA, 26 bp, Escherichia coli
  source 1..26: mol_type = other DNA
agcccggggatttcacatctgactta

SEQ ID NO: 60 - DNA, 14 bp, Escherichia coli
  source 1..14: mol_type = other DNA
cggagggtgcaagc

SEQ ID NO: 61 - DNA, 23 bp, Streptococcus sp.
  source 1..23: mol_type = other DNA
gctacacacgtgctacaatggtt

SEQ ID NO: 62 - DNA, 24 bp, Streptococcus sp.
  source 1..24: mol_type = other DNA
cagcctacaatccgaaccgagatt

SEQ ID NO: 63 - DNA, 24 bp, Streptococcus sp.
  source 1..24: mol_type = other DNA
cagcctacaatccgaaccgagact

SEQ ID NO: 64 - DNA, 21 bp, Streptococcus sp.
  source 1..21: mol_type = other DNA
cgcaagccggtgacggcaagc

SEQ ID NO: 65 - DNA, 20 bp, Lactobacillus crispatus
  source 1..20: mol_type = other DNA
gggaacctgccccatagtct

SEQ ID NO: 66 - DNA, 22 bp, Lactobacillus crispatus
  source 1..22: mol_type = other DNA
tgatcatgcgatctgctttctt

SEQ ID NO: 67 - DNA, 18 bp, Lactobacillus crispatus
  source 1..18: mol_type = other DNA
ataccacttggaaacagg

SEQ ID NO: 68 - DNA, 26 bp, Lactobacillus gasseri
  source 1..26: mol_type = other DNA
ctgggataacacctggaaacagatgc

SEQ ID NO: 69 - DNA, 28 bp, Lactobacillus gasseri
  source 1..28: mol_type = other DNA
tgcatcattgccttggtaagccgttacc

SEQ ID NO: 70 - DNA, 21 bp, Lactobacillus gasseri
  source 1..21: mol_type = other DNA
actctagacatgcgtctagtg

SEQ ID NO: 71 - DNA, 21 bp, Lactobacillus jensenii
  source 1..21: mol_type = other DNA
agtaacgcgtgggtaacctgc

SEQ ID NO: 72 - DNA, 27 bp, Lactobacillus jensenii
  source 1..27: mol_type = other DNA
taaacttctttcatgcgaaagtagctt

SEQ ID NO: 73 - DNA, 21 bp, Lactobacillus jensenii
  source 1..21: mol_type = other DNA
ttaagtctgggataccatttg

SEQ ID NO: 74 - DNA, 21 bp, Candida albicans
  source 1..21: mol_type = other DNA
ccgccagaggtctaaacttac

SEQ ID NO: 75 - DNA, 21 bp, Candida albicans
  source 1..21: mol_type = other DNA
gaaccaagagatccgttgttg

SEQ ID NO: 76 - DNA, 28 bp, Candida albicans
  source 1..28: mol_type = other DNA
caattttttatcaacttgtcacaccaga

SEQ ID NO: 77 - DNA, 19 bp, Candida glabrata
  source 1..19: mol_type = other DNA
tggtgggtgttctgcagtg

SEQ ID NO: 78 - DNA, 20 bp, Candida glabrata
  source 1..20: mol_type = other DNA
ttgcatccactgggagaact

SEQ ID NO: 79 - DNA, 25 bp, Candida glabrata
  source 1..25: mol_type = other DNA
ctgggagtgtgcgtggatctctcta

SEQ ID NO: 80 - DNA, 22 bp, Candida tropicalis
  source 1..22: mol_type = other DNA
agcaatacgctaggtttgtttg

SEQ ID NO: 81 - DNA, 23 bp, Candida tropicalis
  source 1..23: mol_type = other DNA
cctgatttgaggtcaaagttatg

SEQ ID NO: 82 - DNA, 28 bp, Candida tropicalis
  source 1..28: mol_type = other DNA
acgtggaaacttattttaagcgacttag

SEQ ID NO: 83 - DNA, 23 bp, Candida parapsilosis
  source 1..23: mol_type = other DNA
gcctgccagagattaaactcaac

SEQ ID NO: 84 - DNA, 20 bp, Candida parapsilosis
  source 1..20: mol_type = other DNA
cttcatcgatgcgagaacca

SEQ ID NO: 85 - DNA, 23 bp, Candida parapsilosis
  source 1..23: mol_type = other DNA
taatgtcaaccgattatttaata

SEQ ID NO: 86 - DNA, 19 bp, Candida kefyr
  source 1..19: mol_type = other DNA
cagggctgcgtgtcaagtc

SEQ ID NO: 87 - DNA, 23 bp, Candida kefyr
  source 1..23: mol_type = other DNA
gcgcaaacctaagacgtagatgt

SEQ ID NO: 88 - DNA, 16 bp, Candida kefyr
  source 1..16: mol_type = other DNA
atggactcgactcttg

SEQ ID NO: 89 - DNA, 17 bp, Candida krusei
  source 1..17: mol_type = other DNA
gctgcgactcgcctgaa

SEQ ID NO: 90 - DNA, 21 bp, Candida krusei
  source 1..21: mol_type = other DNA
ttgtctcgcaacactcgctct

SEQ ID NO: 91 - DNA, 13 bp, Candida krusei
  source 1..13: mol_type = other DNA
agcgaagctggcc

SEQ ID NO: 92 - DNA, 22 bp, Candida dubliniensis
  source 1..22: mol_type = other DNA
ccctgcctgccgccagaggaca

SEQ ID NO: 93 - DNA, 28 bp, Candida dubliniensis
  source 1..28: mol_type = other DNA
atgcgagaaccaagagatccgttgttga

SEQ ID NO: 94 - DNA, 27 bp, Candida dubliniensis
  source 1..27: mol_type = other DNA
caaattttttataaacttgtcacgaga